(19) United States
(12) Patent Application Publication
(10) Pub. No.: US 2007/0061937 A1
(43) Pub. Date: Mar. 22, 2007

(54) Method and apparatus for aerodynamic hat brim and hat

(76) Inventor: Dennis Wood Curle, Norco, CA (US)

Correspondence Address: Dennis Wood Curle, P.O. Box 7040, Norco, CA, 92860, US

(21) Appl. No.: 11/220,868

(22) Filed: Sep. 6, 2005

(51) Int. Cl. A61F 9/00

(52) U.S. Cl. 2/12

(57) ABSTRACT

An aerodynamic cap brim includes a first and a second outer portion, a first and a second inner portion. The inner portion including a cut-out portion and an airfoil portion. The aerodynamic cap further including a first coupling portion and a second coupling portion. The first coupling portion and the second coupling portion each adapted to couple to a cap. A method includes attaching an aerodynamic cap brim to a cap. The aerodynamic cap brim includes a first and a second outer portion, a first and a second inner portion, where the inner portion includes a cut-out portion and an airfoil portion. A first connecting portion and a second connecting portion where the first connecting portion and the second connecting portion connect to the cap.

### BACKGROUND

1. Field

The embodiments relate to hat brims and hats, and more particularly to methods and apparatus of an aerodynamic hat brim and hat with an aerodynamic hat brim.

2. Description of the Related Art

Hats and caps have been around for a long time. Hats protect people from weather, sun; are stylish, and cover hair and bald spots. Typical caps, such as baseball caps, tighten around a person's head by being tightly fitted, snap couplers, hook and loop fasteners, belts, straps, etc. Most of these ways for attaching the cap tightly to a person's head help try to keep the cap on when a person runs (e.g., in a baseball game), on windy days, etc. These typical caps, however, have a brim that causes lift when a strong wind or wind created by fast movement (e.g., when a person is: on a fast boat, personal watercraft, motorcycle, convertible automobile, etc.). The lift blows the cap off a person's head. The reason for this is that the brim of a basic cap is hard plastic that is covered by fabric that is stitched through the brim. Because the brim is curved and a solid piece, the wind has no where to escape and lifts the cap by the brim, which pulls the cap off a person's head.

### SUMMARY

One embodiment includes an aerodynamic cap brim. The aerodynamic cap brim includes a first and a second outer portion; a first and a second inner portion, the inner portion including a cut-out portion and an airfoil portion; and a first coupling portion and a second coupling portion. The first coupling portion and the second coupling portion each adapted to couple to a cap.

Another embodiment includes a cap. An aerodynamic cap brim is attached to the cap. The aerodynamic cap brim includes: a first outer portion and a second outer portion; a first inner portion and a second inner portion. The inner portion includes a cut-out portion and an airfoil portion. And a first coupling portion and a second coupling portion. The first coupling portion and the second coupling portion each couple the aerodynamic cap brim to the cap.

Still another embodiment includes a method of forming a mold for an aerodynamic cap brim. The mold to form the aerodynamic cap brim including: a first and a second outer portion; a first and a second inner portion, the inner portion including a cut-out portion and an airfoil portion; and a first coupling portion and a second coupling portion. The method further includes injecting a material into the mold. The material enters the mold as a liquid and then hardens forming the aerodynamic cap brim. Removing the aerodynamic cap brim from the mold. And attaching the aerodynamic cap brim to a cap. The first coupling portion and the second coupling portion each couple to the cap.

Yet another embodiment includes a method of forming a mold for an aerodynamic cap brim, the mold to form the aerodynamic cap brim including a first and a second outer portion; a first and a second inner portion, the inner portion including a cut-out portion and an airfoil portion; and a first coupling portion and a second coupling portion. The method further including injecting a material into the mold. The material enters the mold as a liquid and then hardens forming the aerodynamic cap brim. And removing the aerodynamic cap brim from the mold.

And still another embodiment includes a method of attaching an aerodynamic cap brim to a cap. The aerodynamic cap brim includes a first and a second outer portion; a first and a second inner portion, the inner portion including a cut-out portion and an airfoil portion; and a first coupling portion and a second coupling portion. The first coupling portion and the second coupling portion couple to the cap.

### BRIEF DESCRIPTION OF THE DRAWINGS

The embodiments are illustrated by way of example, and not by way of limitation, in the figures of the accompanying drawings and in which like reference numerals refer to similar elements and in which:

FIG. 1 illustrates one embodiment of an aerodynamic hat brim.

FIG. 1A illustrates a cut-through view of the embodiment illustrated in FIG. 1.

FIG. 2 illustrates a front view of the embodiment illustrated in FIG. 1.

FIG. 3 illustrates a rear view of the embodiment illustrated in FIG. 1.

FIG. 4 illustrates still a left side view of the embodiment illustrated in FIG. 1.

FIG. 5 illustrates still a right side view of the embodiment illustrated in FIG. 1.

FIG. 6 illustrates a top view of the embodiment illustrated in FIG. 1.

FIG. 7 illustrates a bottom view of the embodiment illustrated in FIG. 1.

FIG. 8A illustrates wind airflow deflection over the embodiment illustrated in FIG. 1 coupled to a cap.

FIG. 8B illustrates wind airflow deflection under the embodiment illustrated in FIG. 1 coupled to a cap.

FIG. 9 illustrates an embodiment of a cap having an aerodynamic hat brim.

FIG. 10 illustrates a block diagram of a process of forming an embodiment.

FIG. 11 illustrates a block diagram of a process of forming another embodiment.

### DETAILED DESCRIPTION

The embodiments generally relate to aerodynamic hat brims, hats having an aerodynamic hat brim, methods of forming aerodynamic hat brims and methods of attaching an aerodynamic hat brim to a cap. Referring to the figures, exemplary embodiments of the invention will now be described. The exemplary embodiments are provided to illustrate the invention and should not be construed as limiting the scope of the invention.

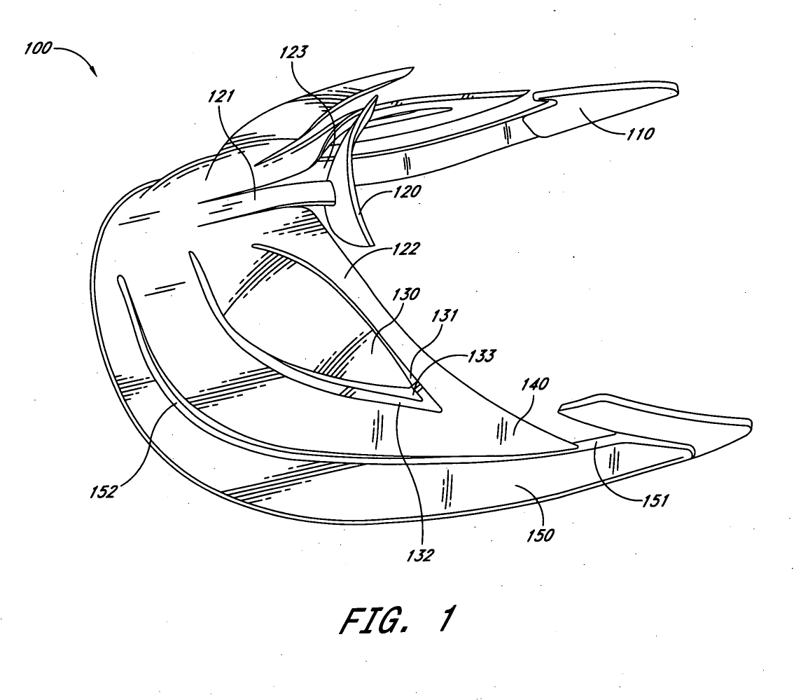

FIG. 1 illustrates an embodiment including aerodynamic cap brim 100. In one embodiment, aerodynamic cap brim 100 includes first and second outer portions 150 and first and second inner portions 140. First and second inner portions 150 each include cut-out portion 133 and airfoil portion130. Aerodynamic cap brim 100 further including first and second coupling portions 110. First and second coupling portions 110 are each adapted to couple to a cap (e.g., a baseball cap). In another embodiment, aerodynamic cap brim is adapted to attach to other headgear that typically has a brim, such as visors. It should be noted that the design of aerodynamic cap brim 100 can be applied to other types of headgear where lift is a problem, such as motorcycle helmets (i.e., the motorcycle helmet sun visor is designed similarly to aerodynamic cap brim 100).

In one embodiment the first outer portion 150 (i.e., left side) couples to second outer portion 150 (i.e., right side) contiguously. In one embodiment first and second outer portions 150 each have a rounded outer edge. In one embodiment first outer and second outer portions 150 each have curved inner edge 151. In one embodiment curved inner edge 151 is angled at about 45 degrees down from the top of aerodynamic cap brim 100. First and second outer portions 150 have a complimentary fit with first and second inner portions 140. In another embodiment, curved inner edge 151 is angled between 30 and 60 degrees down from the top of aerodynamic cap brim 100. In this embodiment, first and second inner portions each have curved first edge 152.

In one embodiment cut-out portion 133 has angled first edge 132 and angled second edge 131. In one embodiment angled first edge 132 is angled at about 45 degrees down from the top of aerodynamic cap brim 100. In this embodiment, angled second edge 131 is angled in a mirrored fashion about 45 degrees down from the top (from the back) of aerodynamic cap brim 100. In another embodiment angled first edge 132 is angled between 30 and 60 degrees down from the top of aerodynamic cap brim 100. In this embodiment, angled second edge 131 is angled in a mirrored fashion between 30 and 60 degrees down from the top (from the back) of aerodynamic cap brim 100.

In one embodiment airfoil portion 130 has an angled first edge (front) and an angled second edge (back) that are complimentary fitted with angled first edge 132 and angled second edge 131, respectively, of cutout portion 133. In one embodiment back portion 122 of inner portion 140 has a rounded edge. In another embodiment, back portion 122 has an angled edge that is about 45 degrees angled up from the bottom of aerodynamic cap brim 100 towards the top (back side). In yet another embodiment, back portion 122 has an angled edge that is between 30 and 60 degrees angled up from the bottom of aerodynamic cap brim 100 towards the top (back side).

In one embodiment, aerodynamic cap brim 100 is made of a plastic or similar material. In one embodiment, aerodynamic cap brim 100 is made of material similar or the same as typical cap brims. In this embodiment, aerodynamic cap brim 100 is sanded and cut. In one embodiment, paint can be applied to the hard plastic material. In another embodiment, aerodynamic cap brim 100 is made from injection molding. In other embodiments, similar materials can be used that can flex, i.e., materials that are not too rigid. In one embodiment, processed leather is used to make aerodynamic cap brim 100.

In one embodiment aerodynamic cap brim includes third coupling portion 120. In this embodiment third coupling portion 120 is connected to center portion 121. Third coupling portion 120 is adapted to couple to a cap. In one embodiment, when aerodynamic cap brim is coupled to a cap, as only first and second coupling portions 110 and third coupling portion 120 is coupled to the cap, a space exists between the cap and back portion 122. In one embodiment the space is ¼ of an inch or less. It should be noted that a larger or slightly larger space than ¼ inch will not effect the wind deflection capability of aerodynamic cap brim 100. In another embodiment space 123 exists between center portion 121 and back portion 122. In this embodiment, this space is approximately ⅛ to ¼ of an inch. In another embodiment, this space is approximately 1/16 to ½ of an inch. It should be noted that aerodynamic cap brim 100 is sized as typical cap brims in the industry. It should be noted, however, that the size of aerodynamic cap-brim can be larger or smaller than typical cap brims in the industry without effecting the embodiments.

In one embodiment first, second (110) and third (120) coupling portions are sewn into a cap. In another embodiment first, second (110) and third (120) coupling portions are riveted to a cap. It should be noted that first, second (110) and third (120) coupling portions can be attached in other ways as well without deviating from the scope of the embodiments. In one embodiment, first and second coupling portions (110) have a hooked end. In other embodiments, other shapes can be used, such as straight, curved, square, etc. In yet other embodiments, first and second coupling portions (110) have through-holes for coupling to a cap.

FIG. 1A illustrates a cut through view of aerodynamic cap brim 100. This view is looking at the inside of the right hand portion of aerodynamic cap brim 100 when cut in half.

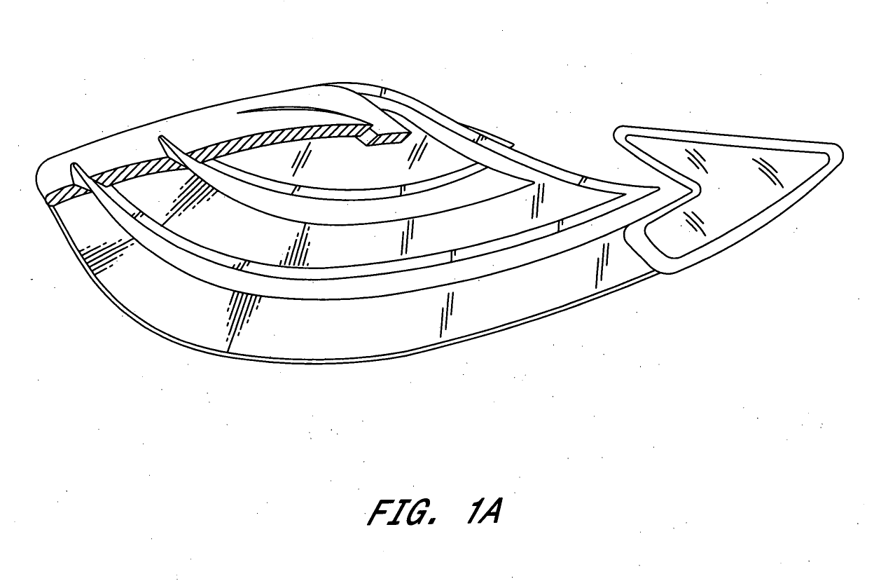

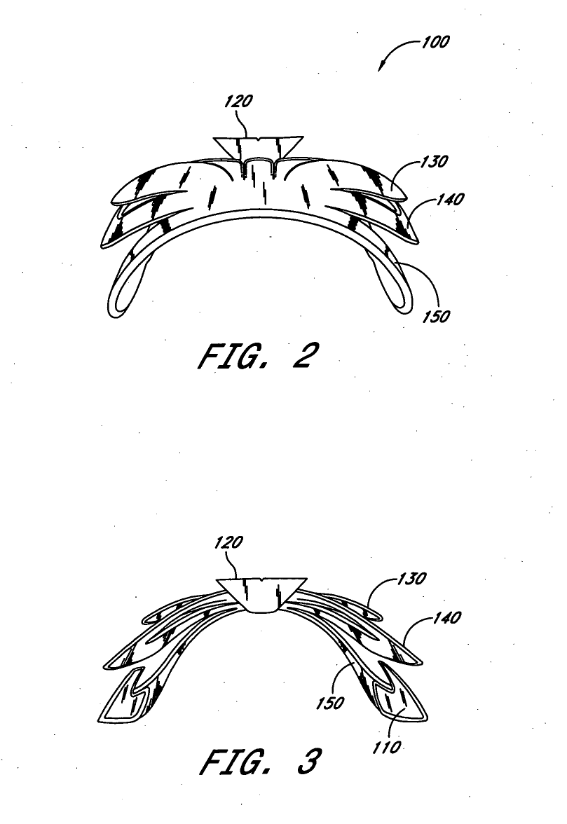

FIG. 2 illustrates a front side view of aerodynamic cap brim 100. FIG. 3 illustrates a rear side view of aerodynamic cap brim 100. In these illustrations, the spacing apart of first and second outer portion 150, first and second inner portions 140, and airfoil portions 130 are exaggerated or forced. When no force is placed on aerodynamic cap brim 100, first and second outer portions 150, first and second inner portions 140, and airfoil portions 130 are all flush together in a complimentary fitment.

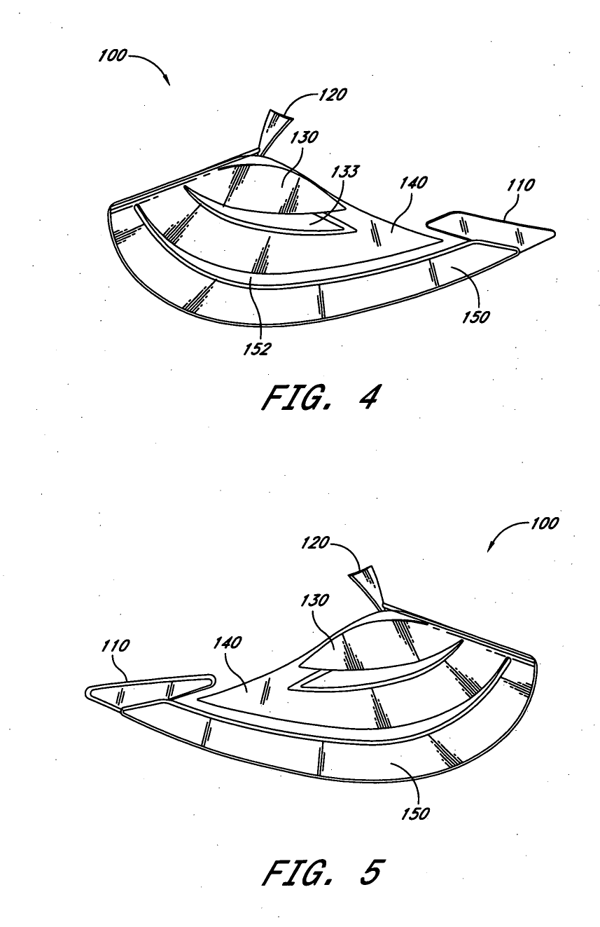

FIG. 4 illustrates a left-hand side view of aerodynamic cap brim 100. FIG. 5 illustrates a right-hand side view of aerodynamic cap brim 100. In these illustrations, the spacing apart of first and second outer portion 150, first and second inner portions 140, and airfoil portions 130 are exaggerated or forced. When no force is placed on aerodynamic cap brim 100, first and second outer portions 150, first and second inner portions 140, and airfoil portions 130 are all flush together in a complimentary fitment.

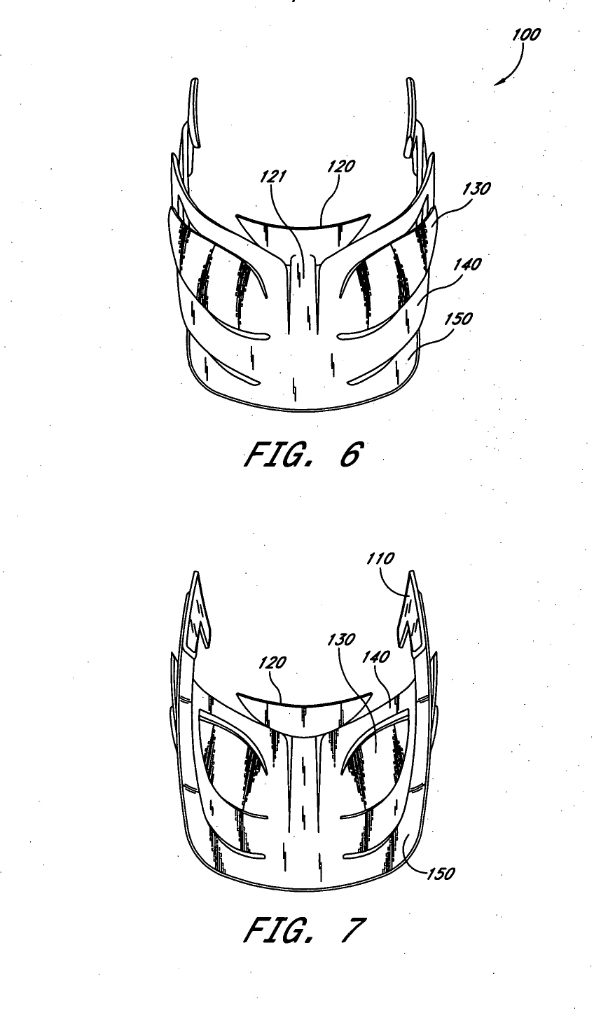

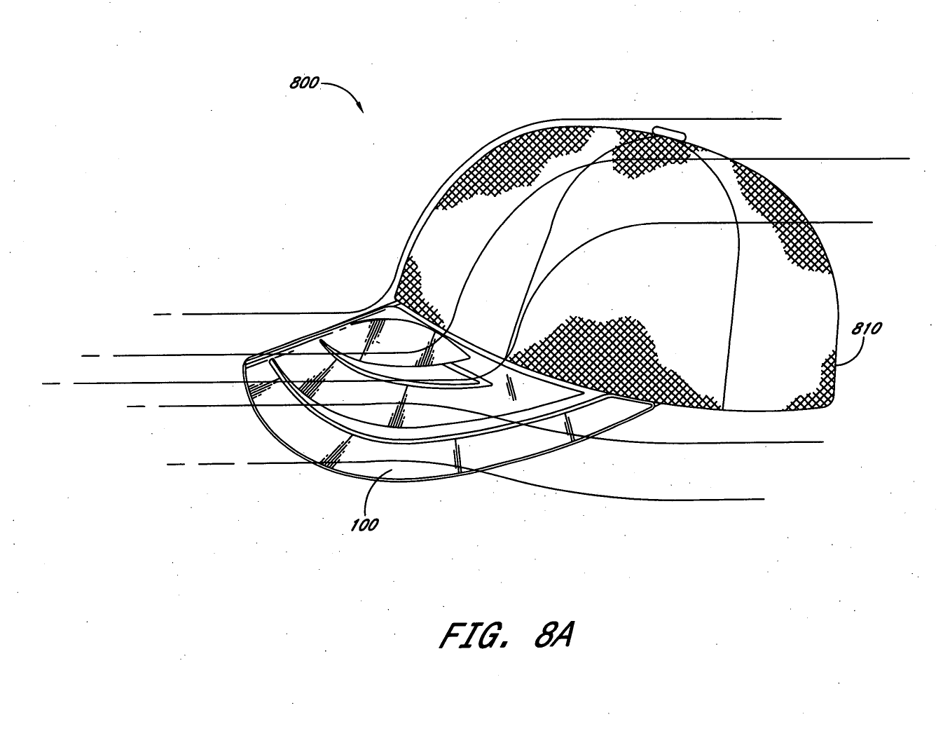

FIG. 6 illustrates a top view of aerodynamic cap brim 100. FIG. 7 illustrates a bottom view of aerodynamic cap brim 100. In these illustrations, the spacing apart of first and second outer portion 150, first and second inner portions 140, and airfoil portions 130 are exaggerated or forced. When no force is placed on aerodynamic cap brim 100, first and second outer portions 150, first and second inner portions 140, and airfoil portions 130 are all flush together in a complimentary fitment.

Aerodynamic cap brim 100 redirects forced air (e.g., from natural wind, wind resistance caused from moving, created air (e.g., from a fan, or compressed air), etc., from an attached cap to prevent the attached cap from lifting off a person's head when attached thereto. In this embodiment, forced air (i.e., natural wind, unnatural wind, wind resistance, etc.) causes first and second inner portions 140 and airfoil portions 130 to flex upward when directed under aerodynamic cap brim 100. When first and second inner portions 140 and airfoil portions 130 flex upward, the air is re-directed around the sides (with some air redirected over) the cap. This re-direction eliminates lift, which would otherwise cause the cap to be forced off a person's head.

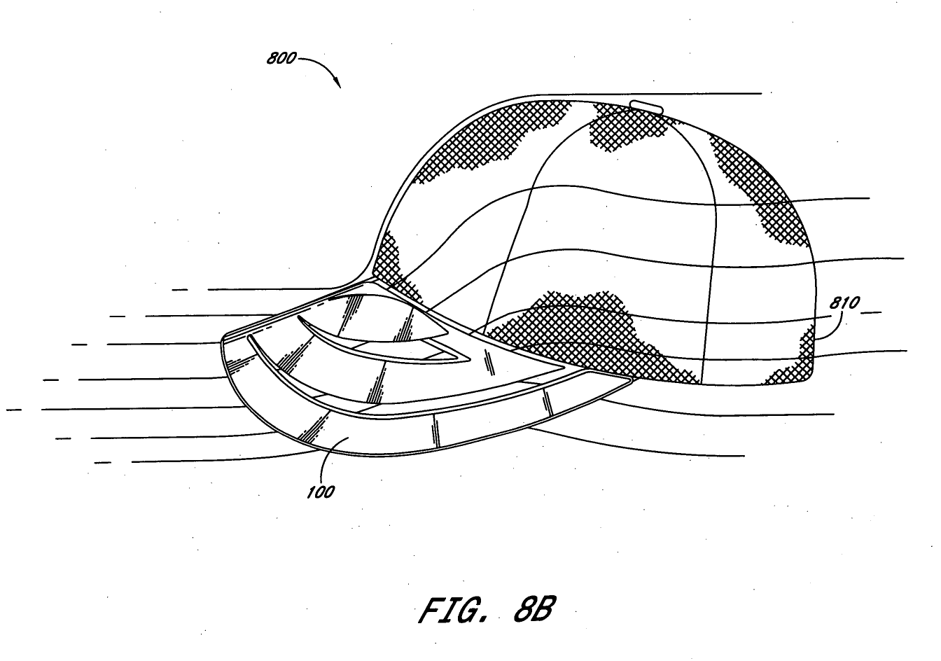

Typically when air is forced downward on a typical cap and typical cap brim, no problem exists with the cap being forced off a persons head. However, a person's neck can get forced downward, which can cause strain over time. Aerodynamic cap brim 100 allows some air to flow through the openings between airfoil portions 130 and cut-out portions 133, and between first and second inner portions 140 and first and second outer portions 150. This flow through allows the force from forced air (i.e., natural or unnatural) to be reduced, which reduces strain on a person wearing aerodynamic cap brim 100 attached to a cap. FIG. 8A illustrates re-directed air directed over aerodynamic cap brim 100. FIG. 8B illustrates re-directed air directed under aerodynamic cap brim 100.

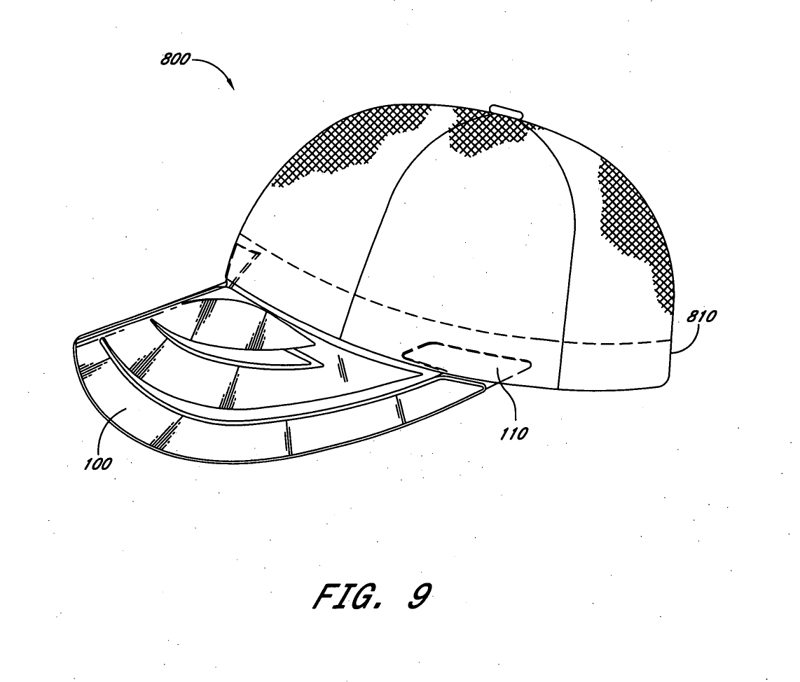

FIG. 9 illustrates an embodiment including cap 810 with aerodynamic cap brim 100 attached to cap 810. In one embodiment, cap 810 is a typical cap and has typical means for attachment to a person's head. In this embodiment, cap 810 is attached to a person's head by hook and loop fasteners, plastic/metal snaps, belts, fitted wear, etc. FIG. 9 illustrates the placement of first and second coupling portions 110 and third coupling portion 120 when attached to cap 810.

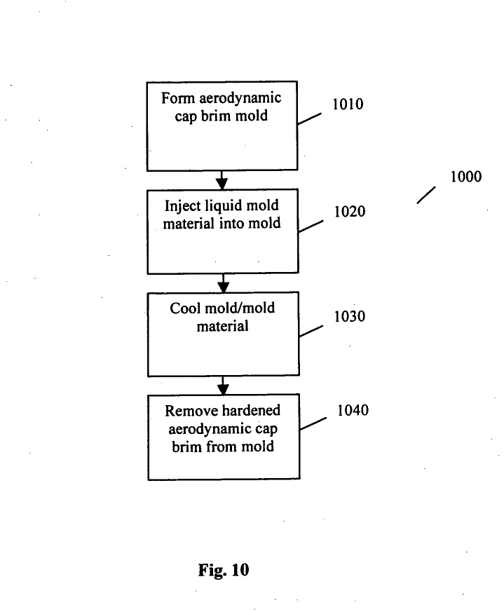

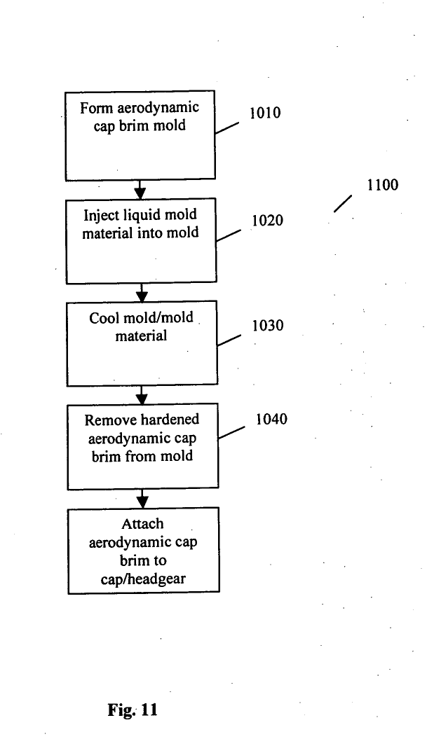

FIG. 10 illustrates a block diagram of a process of forming aerodynamic cap brim 100. Process 1000 begins with block 1010. In block 1010, an injection mold is formed for an aerodynamic cap brim (i.e., aerodynamic cap brim 100). In process 1000, typical mold forming techniques known in the industry are used to form the mold for the design of aerodynamic cap brim 100.

Process 1000 continues with block 1020. In block 1020, liquid material is injected into the formed mold. Typically, a hot or melted material, such as melted plastic or similar materials are heated to a liquid form. The liquid is injected into the mold where it flows to completely fill the mold cavity.

Process 1000 continues with block 1030. In block 1030, the mold material is cooled. Typical cooling techniques known in the industry can be used, whether artificial cooling or natural cooling. When the liquid material cools, the material hardens.

Process 1000 continues with block 1040 where the cooled and hardened aerodynamic cap brim (i.e., aerodynamic cap brim 100) is removed from the mold. In some embodiments, once the aerodynamic cap brim 100 is removed from the mold, the aerodynamic cap brim may need to be trimmed or sanded to remove rough edges or excess mold material in order to smooth out aerodynamic cap brim 100. In one embodiment, once the aerodynamic cap brim is formed and cleaned of excess material, aerodynamic cap brim 100 can be ornamentally embellished with paint, or sealants.

FIG. 11 illustrates a block diagram of process 1100. Process 1100 is similar to process 1000 for blocks 1010, 1020, 1030 and 1040. In process 1100, after block 1040 is completed and the aerodynamic cap brim is cleaned of any excess material, process 1100 continues with block 1110. In block 1110, aerodynamic cap brim 100 is then attached to a cap. Typical machinery known in the industry can be used for attaching aerodynamic cap brim 10 to a cap or headgear (e.g., cap 810, a visor, etc.). In one embodiment, aerodynamic cap brim 100 is attached to a cap by sewing first and second (110) and third (120) coupling portions to the cap (e.g., directly sewing the coupling portions or sewing locking pockets around the coupling portions). In other embodiments, machines for riveting (e.g., plastic or metal/metal alloy rivets) are used for attaching aerodynamic cap brim 100 to a cap.

In one embodiment, preformed aerodynamic cap brims are already formed and delivered to a cap/headgear maker. In this embodiment, aerodynamic cap brim 100 is then attached to a cap/headgear. In this process embodiment, a plurality of aerodynamic cap brims 100 are delivered as ordered and individual cap/headgear makers can attach their cap/headgear as desired.

The length, diameter, size, etc. of the elements of the above embodiments can be made to suit any of the above embodiments for different sized uses (e.g., larger or smaller caps/aerodynamic cap brims, thicker or thinner aerodynamic cap brims 100).

The embodiments are illustrated by way of example and not by way of limitation in the figures of the accompanying drawings in which like references indicate similar elements. It should be noted that references in the specification to “an embodiment,” “one embodiment,” “some embodiments,” or “other embodiments” means that a particular feature, structure, or characteristic described in connection with the embodiments is included in at least some embodiments, but not necessarily all embodiments, of the invention. The various appearances “an embodiment,” “one embodiment,” or “some embodiments,” are not necessarily all referring to the same embodiments. If the specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

1. An apparatus comprising:
an aerodynamic cap brim, the aerodynamic cap brim comprising:
a first and a second outer portion;
a first inner portion including a first cut-out portion and a first airfoil portion, the first airfoil portion covers the first cut-out portion;
a second inner portion including a second cut-out portion and a second airfoil portion, the second airfoil portion covers the second cut-out portion; and
a first coupling portion and a second coupling portion,
wherein the first coupling portion and the second coupling portion each adapted to couple to a cap.

2. The apparatus of claim 1, wherein the first outer portion couples to the second outer portion contiguously.

3. The apparatus of claim 1, wherein the first outer portion and the second outer portion each have a curved inner edge.

4. The apparatus of claim 1, wherein the first inner portion and the second inner portion each have a curved first edge.

5. The apparatus of claim 1, wherein the first and second cut-out portions each have an angled first edge and an angled second edge.

6. The apparatus of claim 1, wherein the first and second airfoil portions each have an angled first edge and an angled second edge.

7. The apparatus of claim 6, wherein the angled first edge of the first and second cut-out portions is complimentary to the angled first edge of the first and second airfoil portions.

8. The apparatus of claim 6, wherein the angled second edge of the first and second cut-out portions is complimentary to the angled second edge of the first and second airfoil portions.

9. The apparatus of claim 1, further comprising a third coupling portion, the third coupling portion coupled to a center portion, wherein the third coupling portion adapted to couple to the cap.

10. An apparatus comprising:
a cap;
an aerodynamic cap brim coupled to the cap, the aerodynamic cap brim comprising:
a first outer portion and a second outer portion;
a first inner portion including a first cut-out portion and a first airfoil portion, the first airfoil portion covers the first cut-out portion;
a second inner portion including a second cut-out portion and a second airfoil portion, the second airfoil portion covers the second cut-out portion; and
a first coupling portion and a second coupling portion;
wherein the first coupling portion and the second coupling portion each couple the aerodynamic cap brim to the cap.

11. The apparatus of claim 10, wherein the first outer portion and the second outer portion each have a curved inner edge.

12. The apparatus of claim 10, wherein the first inner portion and the second inner portion each have an angled first edge.

13. The apparatus of claim 10, wherein the first and second cut-out portions each have an angled first edge and an angled second edge.

14. The apparatus of claim 10, wherein the first and second airfoil portions each have an angled first edge and an angled second edge.

15. The apparatus of claim 14, wherein the angled first edge of the first and second cut-out portions is complimentary to the angled first edge of the first and second airfoil portions.

16. The apparatus of claim 14, wherein the angled second edge of the first and second cut-out portions is complimentary to the angled second edge of the first and second airfoil portions.

17. The apparatus of claim 10, further comprising a third coupling portion, the third coupling portion coupled to a center portion, wherein the third coupling portion adapted to couple to the cap.

18. The apparatus of claim 10, the aerodynamic cap brim redirects forced air to avoid lift.

19. A method comprising:
forming a mold for an aerodynamic cap brim, the mold to form the aerodynamic cap brim comprising:
a first and a second outer portion;
a first inner portion including a first cut-out portion and a first airfoil portion, the first airfoil portion covers the first cut-out portion;
a second inner portion including a second cut-out portion and a second airfoil portion, the second airfoil portion covers the second cut-out portion and
a first coupling portion and a second coupling portion;
injecting a material into the mold, where the material enters the mold as a liquid and then hardens forming the aerodynamic cap brim;
removing the aerodynamic cap brim from the mold; and
attaching the aerodynamic cap brim to a cap,
wherein the first coupling portion and the second coupling portion each couple to the cap.

20. The method of claim 19, wherein the first and second cut-out portions are each formed with an angled first edge and an angled second edge.

21. The method of claim 19, wherein the first and second airfoil portions are each formed with an angled first edge and an angled second edge.

22. The method of claim 19, the aerodynamic hat brim further comprising:
a third coupling portion, the third coupling portion coupled to a center portion, wherein the third coupling portion adapted to couple to the cap.

23. A method comprising:
forming a mold for an aerodynamic cap brim, the mold to form the aerodynamic cap brim comprising:
a first and a second outer portion;
a first inner portion including a first cut-out portion and a first airfoil portion, the first airfoil portion covers the first cut-out portion;
a second inner portion including a second cut-out portion and a second airfoil portion the second airfoil portion covers the second cut-out portion; and
a first coupling portion and a second coupling portion;
injecting a material into the mold, where the material enters the mold as a liquid and then hardens forming the aerodynamic cap brim; and
removing the aerodynamic cap brim from the mold.

24. The method of claim 23, wherein the first and the second cut-out portions are each formed with an angled first edge and an angled second edge.

25. The method of claim 23, wherein the first and second airfoil portions are each formed with an angled first edge and an angled second edge.

26. A method comprising:
attaching an aerodynamic cap brim to a cap, the aerodynamic cap brim comprising:
a first and a second outer portion;
a first and a second inner portion, the inner portion including a cut-out portion and moveable airfoil portion; and
a first inner portion including a first cut-out portion and a first airfoil portion, the first airfoil portion covers the first cut-out portion;
a second inner portion including a second cut-out portion and a second airfoil portion, the second airfoil portion covers the second cut-out portion; and
a first coupling portion and a second coupling portion;
wherein the first coupling portion and the second coupling portion couple to the cap
